(19) United States
(12) Patent Application Publication
(10) Pub. No.: US 2010/0185008 A1
(43) Pub. Date: Jul. 22, 2010

(54) Method for Producing Fenofibrate

(75) Inventor: Valery Dambrin, Chasse Sur Rhone (FR)

Correspondence Address: ADELI & TOLLEN, LLP, 11940 San Vicente Blvd., Suite 100, LOS ANGELES, CA, 90049, US

(73) Assignee: FINORGA, Chasse Sur Rhone (FR)

(21) Appl. No.: 12/667,691

(22) PCT Filed: Jul. 7, 2008
(86) PCT No.: PCT/FR08/00972

§ 371 Date: Jan. 4, 2010

(30) Foreign Application Priority Data
Jul. 5, 2007 (FR) 0704852
Jul. 10, 2007 (FR) 070996

(51) Int. Cl. C07C 69/738

(52) U.S. Cl. 560/52

(57) ABSTRACT

Method for producing fenofibrate from fenofibric acid by in situ preparation of fenofibric acid chloride by means of the action of a chlorinating agent on the acid then by reaction with isopropanol without isolation of the acid chloride.

### FIELD OF THE INVENTION

The present invention relates to a method for preparing fenofibrate.

Fenofibrate, the structure of which is of the formula:

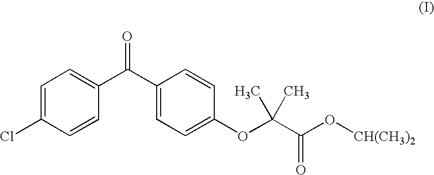

is a product, the hypocholesterolemic and hypolipidemic properties of which are known.

### TECHNOLOGICAL BACKGROUND

The route for accessing this product as well as the products of the families of fibrates has been widely studied.

In British patent application GB 1 539 897 or the corresponding French patent application FR 2 300 552, preparation of fenofibrate is notably proposed by action of the brominated derivative

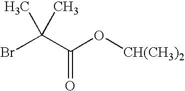

on (4-chlorophenyl)-4-(hydroxyphenyl)-methanone of formula

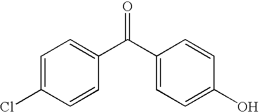

According to the description of these patent applications, it is also possible to prepare fenofibric acid of formula:

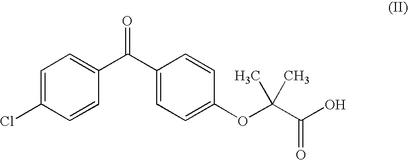

and to then transform it into an ester notably by passing by the preparation of the acid chloride by cold (0-5° C.) action of phosphorus pentachloride and to then react the desired alcohol. According to the described tests, the acid chloride is isolated and recrystallized prior to any other transformation. Fenofibrate is prepared by esterification in a sulfuric acid medium. It is indicated that the preferred method is transesterification.

According to European patent application EP 245 156, it is possible to apply the reaction of the brominated derivative on (4-chlorophenyl)-4-(hydroxyphenyl)-methanone in the presence of excess potassium carbonate relatively to the stoichiometric proportions at a temperature greater than or equal to 120° C. The described method provides interesting yields, however it requires an operation at high temperatures.

### SUMMARY OF THE INVENTION

It has now been found that the preparation of the fenofibrate of formula (I) may be operated starting with fenofibric acid of formula:

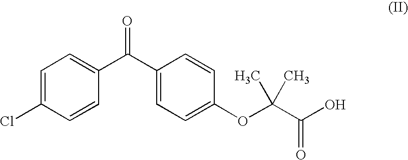

by preparation of fenofibric acid chloride in situ, by action of a chlorination agent on the acid of formula (II), and then by action of isopropanol without isolation of the acid chloride.

According to an embodiment, fenofibric acid chloride is prepared in situ by action of a chlorination agent selected from all known agents allowing such a reaction to be achieved, which do not affect the remainder of the molecule, it is notably selected from thionyl chloride, sulfuryl chloride, oxalyl chloride, carbonyl chloride, phosphorus trichloride or phosphoryl chloride. It is understood that with the chlorination agent, it is possible to simultaneously achieve chlorination and dehydration of fenofibric acid of general formula (II).

Preferably, fenofibric acid chloride is prepared in situ by action of thionyl chloride, sulfuryl chloride, oxalyl chloride or phosphoryl chloride and among these agents, more particularly thionyl chloride.

According to an embodiment, the method according to the invention is operated without adding any solvent.

According to an embodiment, isopropanol is used both as a solvent and as a reagent.

According to an embodiment, the method according to the invention is applied in the presence of a base.

The base may be an alkaline carbonate or hydroxide, in particular an alkaline carbonate such as potassium or sodium carbonate, or sodium or potassium hydroxide; the base may also be an amine, preferably a tertiary amine and among the tertiary amines, notably triethylamine or pyridine or derivatives thereof.

According to an embodiment, the method according to the invention is operated at a temperature from 60 to 90° C.

According to an embodiment, the method according to the invention is applied with excess chlorination agent (thionyl chloride, sulfuryl chloride, oxalyl chloride, carbonyl chloride, phosphorus trichloride or phosphoryl chloride). Preferably it is operated with excess thionyl chloride, sulfuryl chloride, oxalyl chloride or phosphoryl chloride. And more preferably the method according to the invention is operated with excess thionyl chloride.

### DESCRIPTION OF EMBODIMENTS OF THE INVENTION

Preparation of the acid chloride is advantageously carried out in isopropanol, preferably in the presence of a slight excess of chlorination agent, for example in the presence of an excess of thionyl chloride such that the thionyl chloride/fenofibric acid ratio is comprised between 1.1 and 1.5. The temperature is generally comprised between 60 and 90° C., preferably comprised between 80 and 90° C. It is not necessary to add a solvent, isopropanol being used both as a solvent and as a reagent. As soon as it is formed, the acid chloride reacts with isopropanol in order to form the expected fenofibrate, notably at a temperature comprised between 60 and 90° C.

The reaction may be applied in the presence of a base such as a carbonate, such as for example potassium or sodium carbonate, or in the presence of an alkaline hydroxide such as for example sodium or potassium hydroxide; the base is used for neutralizing hydrochloric acid at the end of the reaction for forming the fenofibrate. It is also possible to use an organic base of the amine type, notably a tertiary amine such as for example triethylamine or pyridine or derivatives thereof.

The method according to the invention is particularly advantageous because it provides a highly improved yield and because it may be applied at a moderate temperature. The reaction is fast and appropriate; it does not require recrystallization of the product after treatment. With the method according to the invention, it is thereby possible to obtain fenofibrate with very high yields and purity level (notably a high purity level relatively to the requirements of the European Pharmacopoeia) and furthermore with simplicity of operation and reduced duration of preparation.

Fenofibric acid of formula (II) may be prepared as described in British patent application GB 1,539,897.

The following examples illustrate the present invention.

### EXAMPLE

In a dual-jacket reactor of 250 mL (reactor A), 60 g of fenofibric acid (1 eq.; 0.188 moles) are introduced and 120 mL of isopropanol (2 volumes). The reaction mixture is heated with reflux. The reaction mixture is heterogeneous. To this suspension, are added 29.11 g of thionyl chloride (1.3 eq.; 0.245 moles) over 3 hours. The reaction mixture is homogenized while adding thionyl chloride in order to obtain a yellow solution. At the end of the addition, the reaction medium is maintained under reflux for about 4 hours (the reaction kinetics are followed by HPLC).

In a reactor B, 28.58 g of K₂CO₃ (1.1 eq.; 0.21 mole) are introduced and 120 mL of water (2 volumes). The mixture is heated to 60-65° C.

The contents of reactor A are hot-poured into the reactor B within about 1 hour. The reactor A is rinsed with 15 mL of isopropanol which are transferred to reactor B. The mixture is stirred for a minimum of 5 minutes. Stirring is stopped and the mixture is left to decant. The lower aqueous phase is removed. 134 mL of water are added to the alcoholic phase, while maintaining the temperature of the reaction mass at 60-65° C. The reaction mass is cooled to 50° C. and initiated with a few mg of fenofibrate. The mixture is maintained for about 30 minutes at 50° C. The medium crystallizes. The temperature is lowered to 0-5° C. within 2 hours and then maintained for a minimum of 30 minutes at this temperature (0-5° C.). The mixture is filtered. The cake is washed three times with 70 mL of water. It is dried for one night at 60° C. in a ventilated oven. 65.61 g of dry product are thereby obtained (yield=96.6%).

Analytical results:

HPLC (area %)

Fenofibrate=99.98%

Fenofibric acid=0.02%.

1. A method for preparing the fenofibrate of formula: 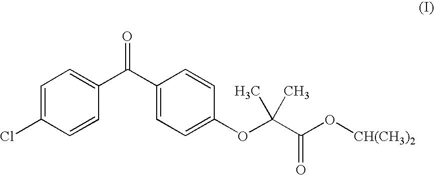
from fenofibric acid of formula 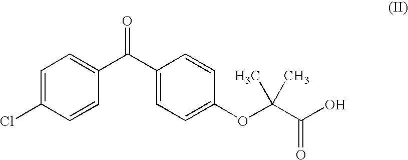
wherein fenofibric acid chloride is prepared in situ, by action of a chlorination agent on the acid of formula (II), and isopropanol is then reacted without isolation of the acid chloride.

2. The method for preparing fenofibrate according to claim 1, wherein the chlorination agent is selected from thionyl choride, sulfuryl chloride, oxalyl chloride, carbonyl chloride, phosphorus trichloride or phosphoryl chloride.

3. The method for preparing fenofibrate according to claim 2, wherein the chlorination agent is thionyl chloride.

4. The method according to claim 1, wherein said preparing is performed without adding any solvent.

5. The method according to claim 1, wherein isopropanol is used both as a solvent and as a reagent.

6. The method according to claim 1, wherein said preparing is performed in the presence of a base.

7. The method according to claim 6, wherein the base is an alkaline carbonate.

8. The method according to claim 6, wherein the base is an alkaline hydroxide.

9. The method according to claim 6, wherein the base is an amine.

10. The method according to claim 1, wherein said preparing is performed at a temperature from 60 to 90° C.

11. The method according to claim 10, wherein said preparing is performed at a temperature from 80 to 90° C.

12. The method for preparing fenofibrate according to claim 1, wherein the chlorination agent is thionyl chloride.
